(19) United States
(12) Patent Application Publication
(10) Pub. No.: US 2003/0050518 A1
(43) Pub. Date: Mar. 13, 2003

(54) Fluorination method

(76) Inventors: Martin Paul Greenhall, Lancashire (GB); Andrew Keith Joel, Lancashire (GB); John Stewart Moilliet, Lancashire (GB)

Correspondence Address: Breiner & Breiner, 115 North Henry Street, PO Box 19290, Alexandria, VA, 22320-0290, US

(21) Appl. No.: 10/129,656

(22) PCT Filed: Sep. 10, 2001
(86) PCT No.: PCT/GB01/04028

(30) Foreign Application Priority Data
Sep. 11, 2000 (GB) 0022166.3

(51) Int. Cl. C07C 25/13

(52) U.S. Cl. 570/147

(57) ABSTRACT

A process is provided for the fluorination of an aromatic fused ring compound which comprises treating the compound with fluorine. A preferred process involves treating a solution of a naphthalene compound in an inert solvent with elemental fluorine, preferably comprised in a gas stream and diluted with an inert gas. The process provides a convenient and simple method by which fluorinated derivatives may be prepared with a high degree of specificity and shows improved yields over methods known from the prior art.

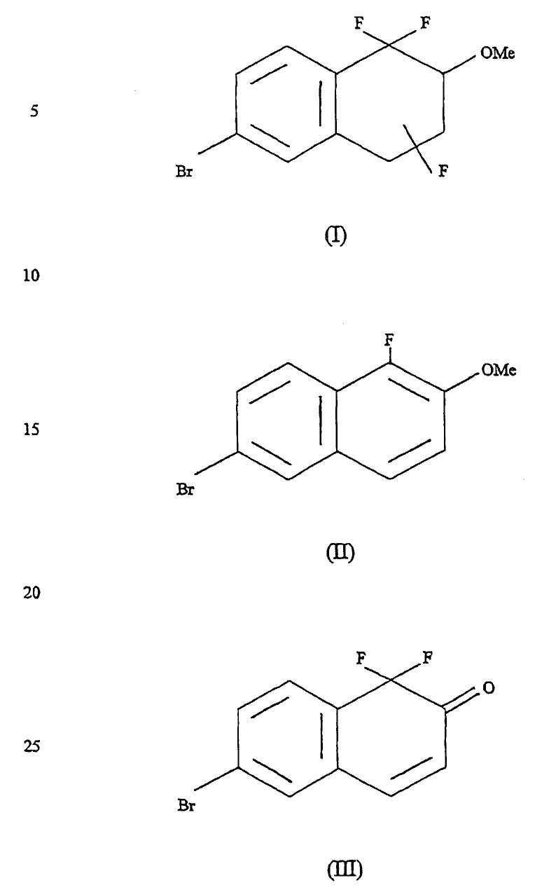

The present invention relates to a method for the fluorination of aromatic organic compounds. More specifically, it concerns a method for the selective direct fluorinaton of fused ring compounds such as derivatives of naphthalene, anthracene, phenanthrene, pyrene and perylene. Of particular interest is the fluorination of the derivatives of anthracene, phenanthrene and, most particularly, naphthalene.

The direct fluorination of aromatic organic compounds has long been an aim of the chemical manufacturing industry. However, for the most part, previous methods have been unsuccessful in providing the desired products in commercially viable yields and at acceptable levels of purity. Consequently, attempted processes have been unattractive and uneconomic.

It is an object of the present invention to provide a process for the fluorination of aromatic compounds—specifically fused ring compounds—which provides good yields of fluorinated compounds showing a high degree of specificity of substitution, allowing for the isolation of fluorinated derivatives in commercially acceptable yields and at high levels of purity.

According to the present invention, there is provided a process for the fluorination of an aromatic fused ring compound, said process comprising treating said compound with fluorine.

Preferably, said aromatic fused ring compound comprises naphthalene, anthracene or phenanthrene or a compound therefo, most preferably naphthalene or its derivative.

Preferably said process is carried out by treating a solution of said compound with elemental fluorine, and said solution is prepared using an inert solvent.

Most preferably, said elemental fluorine is introduced into said solution of said compound as a gas stream, diluted by an inert gas. Typically, said inert gas would be nitrogen and said fluorine gas would be present at a level of 2-30% preferably 5-15%, and most preferably in the region of 10% in the gas mixture. The gas stream would be passed through the reaction mixture at a flow rate of 5-100 ml/min, preferably 15-45 ml/min, and most preferably in the vicinity of 30 ml/min.

A suitable inert solvent would be any solvent in which the aromatic fused ring compound was substantially soluble at the concentrations employed for the reactions, and which did not undergo any reaction with the elemental fluorine under the reaction conditions. Examples of such solvents include formic acid and, more particularly, acetonitrile. It is found that the composition of the reaction products varies with the solvent which is used, so that careful selection is necessary in order to arrive at a desired product. A suitable concentration of the aromatic compound in the said solvent would be in the region of 0.01-2.0 mol/litre, preferably 0.05-1.0 mol/litre and most preferably around 0.1 mol/litre, and the reaction can most conveniently be carried out in a stirred reaction vessel at a temperature of 0-20° C., preferably 5-10° C., with the best results being achieved at temperatures of around 5° C. The reaction time is between 120 and 200 minutes, preferably from 150 to 175 minutes, with the optimum time being about 160 minutes.

The process of the present invention is most suitably applied to naphthalene and its derivatives; specific examples of such compounds which may be fluorinated according to the said process include various mono- and di-substituted naphthalenes wherein the substituent groups include, for example, alkoxy groups, such as methoxy groups, and other halo substituents, typically bromo substituents. Thus, the 1- and 2-methoxy-, the 2,6-dimethoxy-, and the 2bromo-6-methoxy- derivatives of naphthalene have all been successfully fluorinated by the method of the present invention.

Analysis by GC-MS of the reaction mixtures produced when applying the method of the invention to naphthalene an its derivatives indicates that the reactions give rise to mixtures of monofluoro, difluoro and trifluoro compounds, the amounts of the more highly substituted products—not surprisingly—increasing with the duration of the reaction. Separation of these products may be most conveniently achieved by chromatographic techniques, facilitating yields of pure individual products which can be in the region of 40%.

The invention will now be illustrated, though without limitation, by reference to the following examples.

### EXAMPLES

In each case, a naphthalene derivative (0.01 mol) was dissolved in a solvent (100 ml) in a stirred reaction pot. Fluorine (10% in nitrogen) was passed through the solution at a rate of 30 ml/min for 160 minutes, whereupon the reaction was terminated, the reaction mixture was analysed, and the products were separated chromatographically.

Analysis of the reaction mixture was carried out by Gas Chromatograph-Mass Spectroscopy, the reaction mixture being injected without work-up and being run initially for 2 minutes at 150° C., then increased at a rate of 5° C./min., using a non-polar AT-1 capillary column. This analysis took place on completion of the reaction after 160 minutes, and also at the half-way point of the reaction, after 80 minutes.

Retention times and percentage yields of the various products were recorded for fluorination reactions carried out using several napthalene derivatives, as follows:

### Example 1

### 2-Bromo-6-methoxynaphthalene Using Acetonitrile as Reaction Solvent

| | | % content | % content |
|---|---|---|---|
| Retention time/min | Constitution | (80 mins) | (160 mins) |
| 11.1 | Trifluoro deriv. (I) | 3.6 | 29.3 |
| 14.5 | Monofluoro deriv. | 31.1 | 37.3 |
| | (II) | | |
| 15.1 | Starting material | 63.5 | 19.4 |

The products were formed with a high degree of selectivity. The marked increase in the amount of the trifluoro derivative which was present during the second half of the reaction was indicative of the fact that the monofluoro derivative was first formed and then served as a precursor to the trifluoro derivative, which was the expected sequence of events.

Analysis indicated that the trifluoro derivative had lost a degree of unsaturation during the course of the reaction and comprised the tetralin derivative (I) which, on workup, partially defluorinated to yield the α,α-difluoro-β-tetralone compound (III). The monofluoro compound comprised 6-bromo-1-fluoro-2-methoxynaphthalene (II).

### Example 2

### 2-Methoxynaphthalene Using Acetonitrile as Reaction Solvent

| | | % content | % content |
|---|---|---|---|
| Retention time/min | Constitution | (80 mins) | (160 mins) |
| 6.6 | Trifluoro deriv. | 1.5 | 7.9 |
| 8.8 | Monofluoro deriv. | 20.4 | 38.7 |
| 9.0 | Starting material | 75.2 | 44.4 |

Again, the products were formed with a high degree of selectivity, although analysis indicated that a degree of unsaturation may have been lost by the trifluoro derivative.

### Example 3

### 2,6-Dimethoxynaphthalene Using Acetonitrile as Reaction Solvent

| | | % content | % content |
|---|---|---|---|
| Retention time/min | Constitution | (80 mins) | (160 mins) |
| 13.4 | Difluoro deriv. | 3.0 | 17.2 |
| 13.9 | Monofluoro deriv. | 8.9 | 23.5 |
| 14.1 | Starting material | 85.8 | 25.8 |

Selectivity was rather poorer in this case, with four further fluorinated compounds being identified at a level of 3% or greater.

### Example 4

### 1-Methoxynaphthalene Using Acetonitrile as Reaction Solvent

| | | % content | % content |
|---|---|---|---|
| Retention time/min | Constitution | (80 mins) | (160 mins) |
| 7.6 | Monofluoro deriv.1 | 11.4 | 22.2 |
| 8.8 | Monofluoro deriv.2 | 11.2 | 11.8 |
| 8.9 | Starting material | 70.8 | 25.1 |

This reaction again showed a lower degree of specificity than examples 1 and 2, with several products being identified, the major components being two isomeric monofluoro derivatives. It is believed that the second of these derivatives may be more reactive than the first, and undergo further fluorination during the second half of the reaction, thus explaining the low increase in content during this time.

### Example 5

### 2-Bromo-6-methoxynaphthalene Using Formic Acid as Reaction Solvent

| | | % content | % content |
|---|---|---|---|
| Retention time/min | Constitution | (80 mins) | (160 mins) |
| 13.7 | Difluoro deriv.1 | 6.6 | 32.7 |
| 14.1 | Difluoro deriv.2 | 1.2 | 9.8 |
| 14.5 | Monofluoro deriv. | 2.3 | 4.6 |
| 15.0 | Starting material | 86.7 | 51.3 |

The relatively poor solubility of the starting material in the chosen solvent was thought to provide the main reason for the poor level of conversion of the starting material. Initial analytical results suggested that the difluorinated materials were, in fact, the corresponding 2-bromo-6-hydroxy compounds, and that demethylation of the methoxy group had occurred during the reaction. This example, when contrasted with example 1, illustrates the variation in product composition which results from the use of different solvents.

1. A process for the fluorination of an aromatic fused ring compound, said process comprising treating said compound with fluorine.

2. A process as defined in claim 1 wherein said aromatic fused ring compound comprises naphthalene, anthracene, phenanthrene, or a derivative thereof.

3. A process as defined in claim 2 wherein said compound comprises naphthalene or a derivative thereof.

4. A process as defined in any of claims 1-3 wherein said compound is treated with elemental fluorine.

5. A process as defined in any preceding claim wherein said compound is in solution.

6. A process as defined in claim 5 wherein said solution is prepared by dissolving said compound in an inert solvent.

7. A process as defined in claim 5 or 6 wherein said treatment with fluorine comprises passing fluorine through said solution as a gas stream diluted by an inert gas.

8. A process as defined in claim 7 wherein fluorine is present at a level of 2-30% in said gas stream.

9. A process as defined in claim wherein said level is around 10%.

10. A process as defined in any of claims 7-9 wherein said gas stream is passed through said solution at a flow rate of 5-100 ml/min.

11. A process as defined in claim 10 wherein said flow rate is in the vicinity of 30 ml/min.

12. A process as defined in any of claims 6-11 wherein said inert solvent comprises acetonitrile.

13. A process as defined in any of claims 6-11 wherein said inert solvent comprises formic acid.

14. A process as defined in any of claims 5-13 wherein said aromatic fused ring compound is present in said solution at a level in the region of 0.01-2.0 mol/litre.

15. A process as defined in claim 14 wherein said level is around 0.1 mol/litre.

16. A process as defined in any preceding claim wherein said treatment is carried out at a temperature of 0-20° C.

17. A process as defined in claim 16 wherein said temperature is around 5° C.

18. A process as defined in any preceding claim wherein said treatment is carried out for a duration of between 120 and 200 minutes.

19. A process as defined in claim 18 wherein said duration is about 160 minutes.

20. A process as defined in any of claims 3-19 wherein said derivative of naphthalene includes bromo and/or methoxy groups.

21. A process as defined in claim 20 wherein said derivative of naphthalene comprises 1-methoxynaphthalene, 2-methoxynaphthalene, 2,6-dimethoxy-naphthalene or 2-bromo-6-methoxynaphthalene.

22. A fluorinated derivative of an aromatic fused ring compound whenever obtained by a process as defined in any preceding claim.

23. A fluorinated derivative of naphthalene whenever obtained by a process as defined in any of claims 3-22.
